(19) United States
(12) Patent Application Publication
(10) Pub. No.: US 2012/0022397 A1
(43) Pub. Date: Jan. 26, 2012

(54) Needle Set for a Biopsy Device and Related Method

(76) Inventor: Inderjeet S. Jarial, Carmel, IN (US)

(21) Appl. No.: 12/841,328

(22) Filed: Jul. 22, 2010

(51) Int. Cl. A61B 10/02

(52) U.S. Cl. 600/567

(57) ABSTRACT

A needle set is disclosed for biopsy devices including spring-loaded core (SLC) and vacuum assisted spring-loaded core (VASLC) biopsy devices. The needle set comprises a stylet defining an open proximal end, at least two tissue receiving openings at a distal end thereof and a lumen extending from the open proximal end to the at least two tissue receiving openings, wherein the tissue receiving openings are in fluid communication with the lumen. The needle set further comprises a cannula defining an open distal end with a cutting edge formed thereon. The cannula is slidably mounted over the stylet such that the cannula and stylet are movable relative to one another between respective first and second positions. And the cannula is adapted to sever tissue residing in the tissue-receiving openings in a single cutting stroke to obtain at least two biopsy cores.

### FIELD OF THE INVENTION

The present invention generally relates to tissue biopsy devices. More specifically, the invention relates to biopsy needle sets for biopsy devices including spring loaded core (SLC) biopsy devices and vacuum assisted spring loaded core biopsy devices (VASLC).

### BACKGROUND OF THE INVENTION

In the practice of diagnostic medicine it is often necessary or desirable to perform a biopsy, or to sample selected tissue from a living patient for medical evaluation. Cytological and histological studies of the biopsy sample can then be performed as an aid to the diagnosis and treatment of disease. For example, biopsies can be particularly useful in diagnosing and treating various forms of cancer, as well as other diseases in which a localized area of affected tissue can be identified.

Biopsies are routinely performed on tissue using a biopsy device, which employs a working needle set to engage tissue at a predetermined targeted site. The needle set typically includes a stylet defining a tissue piercing tip and tissue receiving opening, which is slidably disposed within the lumen of a surrounding cannula. In some devices, vacuum is provided to assist in drawing tissue into and maintaining said tissue within the tissue receiving opening.

During a biopsy procedure, the biopsy device is positioned within a cavity at the targeted site for the biopsy. The stylet is momentarily driven into the tissue and tissue then prolapses into the tissue opening. The cannula is then advanced along the stylet to cover the tissue opening. This forward movement of the cannula severs the prolapsed tissue to obtain a tissue sample or core, which becomes trapped within tissue opening of the stylet. With the cannula blocking the tissue opening, the biopsy device is then withdrawn from the target site, carrying the sample residing in tissue opening. To collect the biopsy sample, the cannula is once again retracted to expose the tissue opening of the stylet. The procedure may be repeated several times until satisfactory samples have been obtained.

While the above-described biopsy devices are widely used, a two-fold challenge remains in the biopsy field. Namely, the need to obtain sufficient tissue volume and quality tissue cores, regardless of tissue type, to meet the needs of the pathologist so that a conclusive diagnosis can be achieved, while at the same time minimizing treatment time and stress on the patient.

### SUMMARY

In accordance with a first aspect, the present invention is directed to a needle set for use with biopsy and other tissue harvesting devices. The needle set comprises a stylet movable between a first stylet position and a second stylet position. The stylet defines an open proximal end, at least two tissue receiving openings at a distal end thereof and a lumen extending from the open proximal end to the at least two tissue receiving openings. The lumen and tissue receiving openings are configured such that the tissue receiving openings are in fluid communication with the lumen. The needle stylet further comprises a cannula movable between a first cannula position and a second cannula position. The cannula includes an open distal end with a cutting edge formed thereon, and is slidably mounted over the stylet such that the cannula and stylet are movable relative to one another between their respective first and second positions. Having this configuration, the cannula is adapted to sever tissue residing in the tissue-receiving openings in a single cutting stroke from the first cannula position toward the second cannula position to obtain at least two biopsy cores.

In some aspects of the invention the stylet includes either a sharpened tissue piercing tip at its distal end or a blunt tip. Furthermore, the tissue-receiving openings may include a pair of opposing end sections and a pair of side sections extending longitudinally along the stylet between the end sections, wherein at least one end section and/or side section defines a plurality of protrusions adapted to engage tissue residing in the tissue-receiving openings.

In some aspects of the invention, the tissue receiving openings of the stylet are in communication with a vacuum source, allowing vacuum generated by the vacuum source to draw tissue into the tissue receiving openings while the cannula severs the tissue. And in some embodiments of the invention, the vacuum generated by the vacuum source is delivered to the tissue receiving openings through the lumen.

In some aspects of the invention, the stylet and cannula are operatively connected to a drive assembly, which selectively moves the stylet and cannula between their respective first and second positions.

In accordance with another aspect, the present invention is directed to a biopsy device. The biopsy device comprises a needle set mounted to a housing and a drive mechanism operatively connected to the needle set. The needle set includes a stylet movable relative to the housing between a first stylet position and a second stylet position. The stylet defines an open proximal end, at least two tissue receiving openings at a distal end thereof and a lumen extending from the open proximal end to the at least two tissue receiving openings, wherein the tissue receiving openings are in fluid communication with the lumen. The needle set further includes a cannula movable relative to the housing between a first cannula position and a second cannula position, the cannula including an open distal end with a cutting edge formed thereon. The cannula is slidably mounted over the stylet such that the cannula and stylet are movable relative to one another between their respective first and second positions. The drive mechanism is arranged to selectively move the stylet and cannula between their respective first and second positions. Having this configuration, the cannula is adapted to sever tissue residing in the tissue-receiving openings in a single cutting stroke from the first cannula position toward the second cannula position to obtain at least two biopsy cores.

In some aspects of the invention, the biopsy device further comprises a vacuum source in communication with the at least two tissue receiving openings. The vacuum generated by the vacuum source is delivered to the tissue receiving openings to draw tissue into the tissue receiving openings and/or maintain the tissue within the tissue receiving openings, while the cannula severs the tissue. And in some embodiments of the invention, the vacuum generated by the vacuum source is delivered to the tissue receiving openings through the lumen.

In some aspects of the invention, the biopsy device further includes a manually engageable actuator operatively connected to the drive mechanism. The actuator is adapted to selectively actuate the drive mechanism to, in turn, selectively move the stylet and cannula. In some embodiments, the manually engageable actuator is further adapted to actuate the drive mechanism to, in turn, at least one of (i) selectively move the stylet and cannula and (ii) generate vacuum.

In accordance with another aspect, the invention is directed to a method of harvesting multiple biopsy cores from a target site within a patient. The method comprises the steps of: providing a needle set comprising a stylet including an open proximal end, at least two tissue receiving openings at a distal end thereof and a lumen extending from the open proximal end to the at least two tissue receiving openings, and a cannula defining an open distal end with a cutting edge formed thereon, wherein the cannula is slidably mounted over the stylet such that the cannula and stylet are movable relative to one another; introducing the needle set to the target site and exposing the tissue receiving openings to surrounding target tissue and causing surrounding tissue to prolapse into the tissue receiving openings; and advancing the cannula in a single cutting stroke over the tissue receiving openings and substantially simultaneously severing tissue so as to leave a biopsy core within each tissue receiving opening.

In some aspects of the invention, the method further comprises the steps of generating vacuum; and delivering vacuum to the tissue receiving openings to draw in and maintain tissue within the tissue receiving openings while the cannula severs tissue.

Details of one or more implementations of the invention are set forth in the accompanying drawings and in the description below. Further features, aspects, and advantages of the invention will become apparent from the description, the drawings, and the claims.

### BRIEF DESCRIPTION OF DRAWINGS

FIG. 1 is a side elevational view of a needle set according to an embodiment of the invention with the cannula extended.

FIG. 2 is a side elevational view of the needle set of FIG. 1 with the cannula retracted.

FIG. 3 is a cross-sectional view taken along line 3-3 of the needle set shown in FIG. 1.

FIG. 4 is a cross-sectional view taken along line 4-4 of the needle shown in FIG. 2.

FIG. 5 is an end view of the proximal portion of the needle set of FIG. 1.

FIG. 6 is a perspective view of the stylet of the needle set of FIG. 1.

FIG. 7 is a side elevational view of the stylet of FIG. 6.

FIG. 8 is a cross-sectional view taken along line 8-8 of the stylet shown in FIG. 7.

FIG. 9 is an exploded view of the stylet of FIG. 6.

FIG. 10 is a partial side perspective view of the proximal end of the stylet of FIG. 6.

FIG. 11 is a side elevational view of an embodiment of the stylet of FIG. 6 where the tissue-receiving apertures include a plurality of outwardly extending protrusions.

FIG. 12 is a side elevational view of an embodiment of the stylet of FIG. 6 having a blunt tip.

FIG. 13 is a side elevational view of an embodiment of the stylet of FIG. 1, wherein the stylet is a single piece component.

FIG. 14 is a side elevational view of an embodiment of a SLC biopsy device employing the needle set of FIGS. 1-10, with the stylet extending exposing the tissue-receiving openings.

FIG. 15 is a side elevational view of an embodiment of a VASLC biopsy device including a vacuum source employing the needle set of FIGS. 1-10, with the stylet extended exposing the tissue-receiving openings.

Although the drawings represent embodiments of the present invention, the drawings are not necessarily to scale and certain features may be exaggerated in order to better illustrate and explain the present invention. The exemplification set out herein illustrates certain embodiments of the invention, in one, or more forms, and such exemplifications are not to be construed as limiting the scope of the invention in any manner.

### DETAILED DESCRIPTION

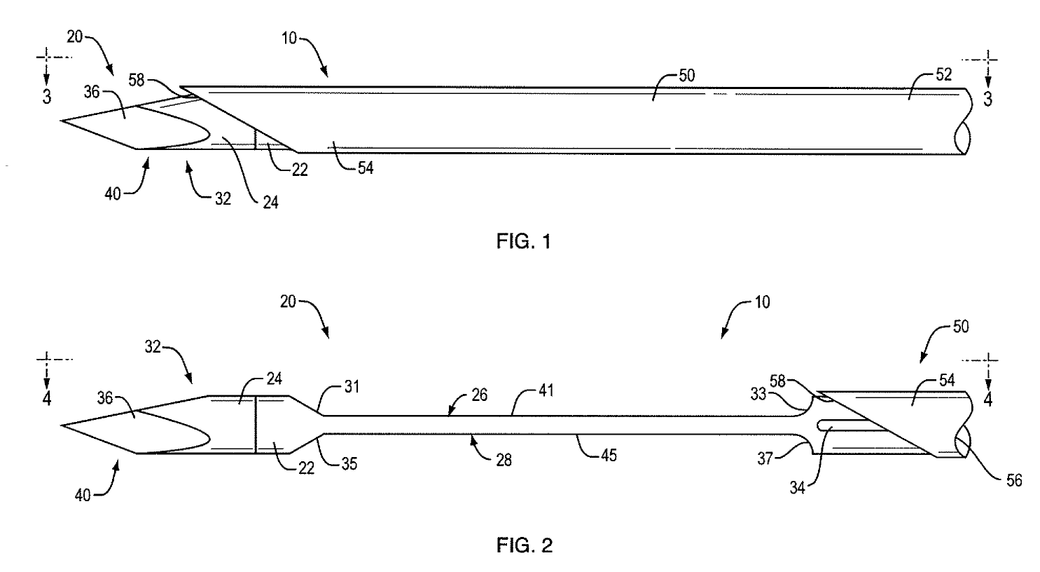

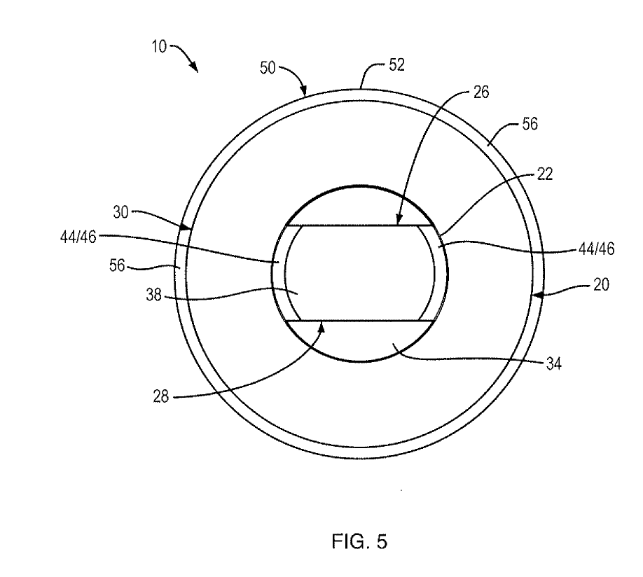

In FIGS. 1-5, a needle set embodying the present invention is indicated generally by the reference numeral 10. Drawing particular attention to FIGS. 1-2, the needle set 10 comprises a stylet 20 (or stylet assembly) slidably disposed within the lumen of a cannula 50 (or cutting cannula) in such a manner that the stylet 20 and cannula 50 are independent movable relative to each other. For example, in one embodiment, the cannula 50 can move or slide over the stylet 20 between a retracted position (FIG. 2) and an extended position (FIG. 1).

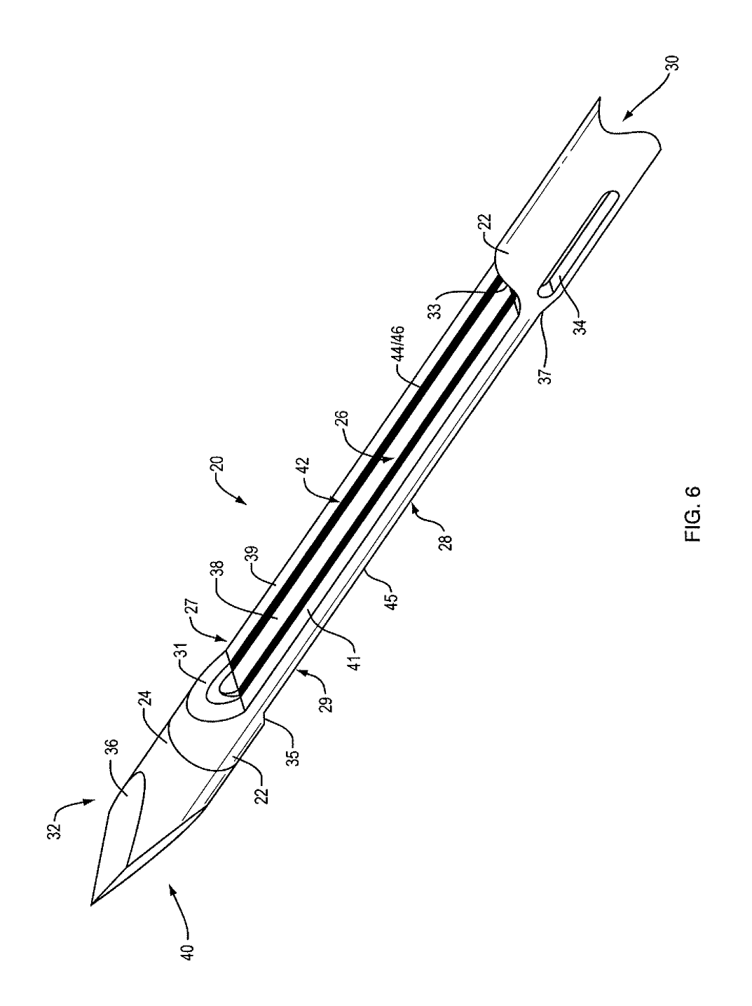

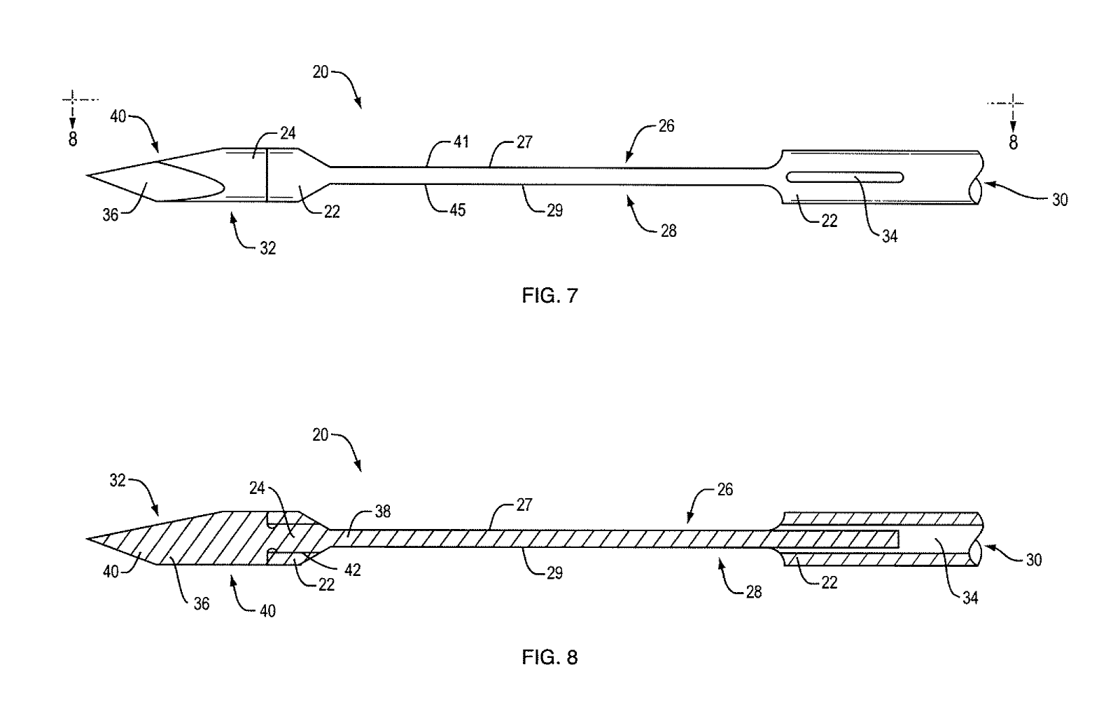

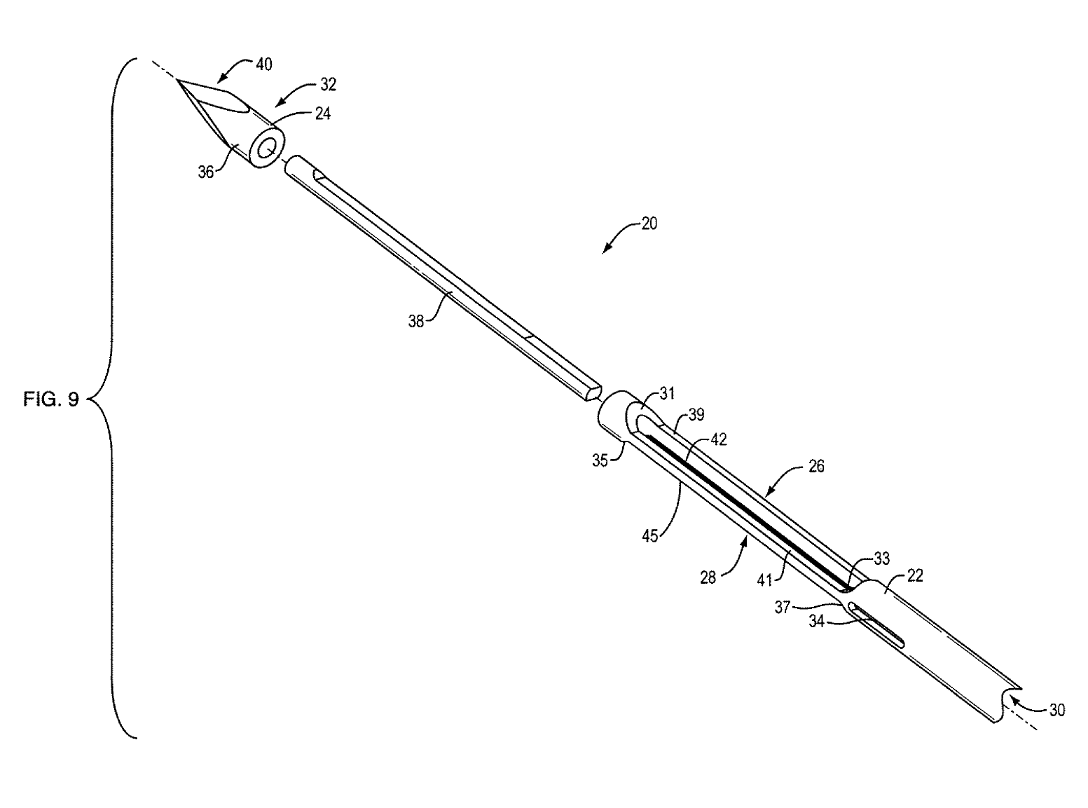

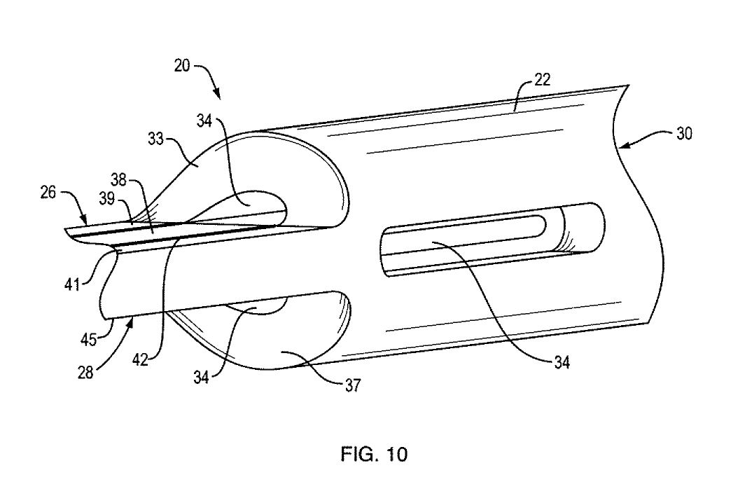

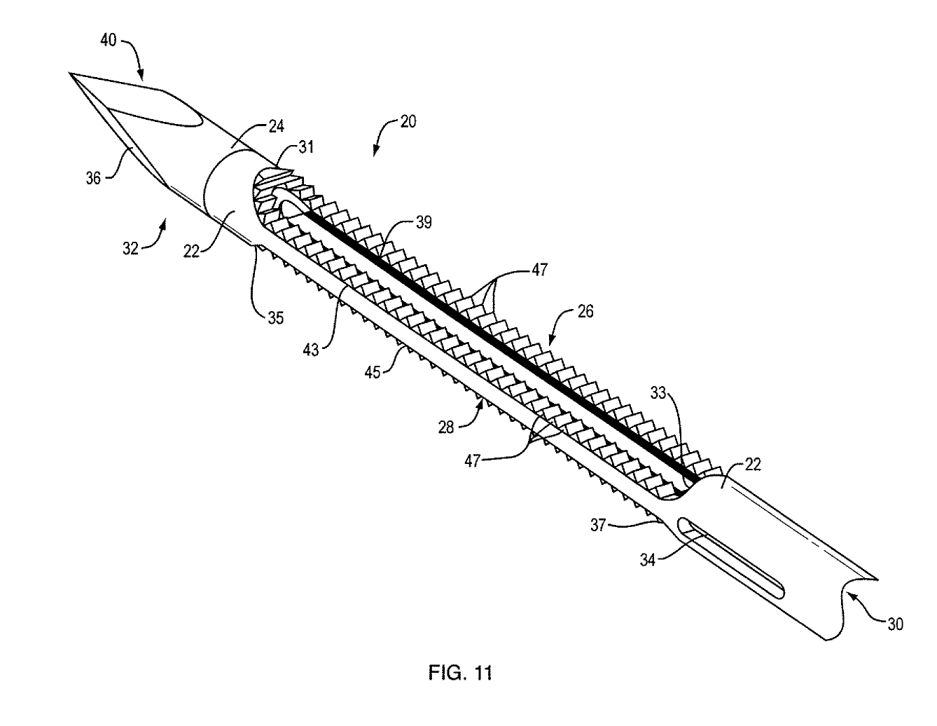

Referring now to FIGS. 6-9, the stylet 20 generally includes a stylet body 22 and a tip body 24 attached thereto. The stylet body 22 defines an open proximal end 30, at least two tissue receiving openings 26, 28 at a distal end 32 thereof, and a lumen 34 extending from the open proximal end 30 to the at least two tissue receiving openings 26, 28. The tissue-receiving openings 26, 28 each include a respective boundary surface 27, 29 defined by a pair of opposing end sections 31, 33, 35, 37 and a pair of side sections 39, 41, 43, 45 (note: side section 43 is shown in FIG. 9 only) extending longitudinally along the stylet 20 between the end sections. As shown, for example, in FIGS. 8 and 10, the lumen 34 and tissue receiving openings 26, 28 are configured such that the tissue receiving openings 26, 28 are in complete unobstructed fluid communication with the lumen 34. The tissue receiving openings 26, 28 are adapted to receive prolapsed tissue from a target tissue site during a biopsy procedure. In the illustrated embodiment, the tissue receiving openings 26, 28 are diametrically opposed; however, it should be noted that the openings could be offset relative to each other along the longitudinal axis of the stylet 20 and/or located at any position relative to each other circumferentially about the perimeter of the stylet 20. Further, in some embodiments, as illustrated, for example, in FIG. 11, the tissue-receiving openings may include a plurality of protrusions 47 extending outwardly from the surface of at least one of the end sections and/or side sections. The protrusions 47 are configured so as to engage tissue residing in the tissue-receiving openings 26, 28 as the cannula 50 is moved distally and/or proximally over the openings. This feature improves cutting performance and prevents severed tissue cores from being compacted when the cannula 50 is retracted so that the clinician can visibly observe the true length and physical quality of the cores upon retrieval. Although the protrusions are illustrated as a series of adjacent serrations or teeth, it should be noted that the protrusions can take on any of numerous shapes, configurations and patterns as recognized by those of ordinary skill in the art. For example, the protrusions could take on a cylindrical or conical shape, or the entire grouping of protrusions could include protrusions of varying pitches, lengths, widths and/or shapes.

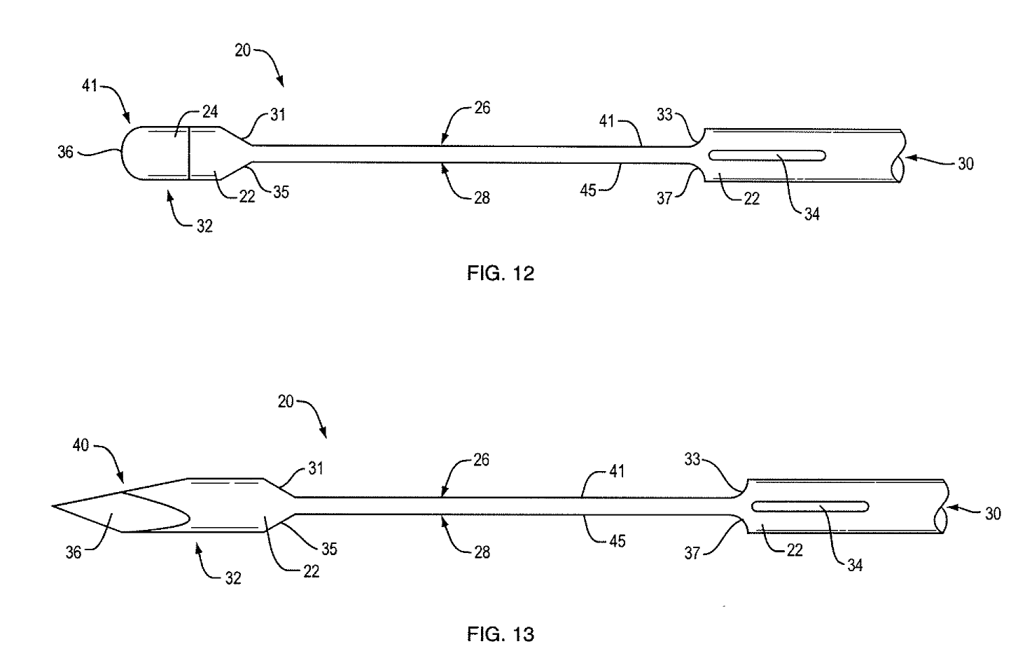

Drawing attention to FIGS. 6, 8 and 9, the tip body 24 of the stylet 20 is shown in further detail. The tip body 24 includes a tip portion 36 (or tip) at its distal outboard end and an elongated shank 38 extending proximally therefrom. In one embodiment, the tip portion 36 is a multi-faceted tissue piercing tip, such as the trocar tip 40 illustrated throughout the figures. However, it should be noted that the tip portion 36 can take on any of numerous configurations known to those of ordinary skill in the art. For example, the tip portion could include one or more sharpened protrusions or blades extending from the faceted surfaces. Also, the tip portion could include less than three facets, more than three facets, or could take the form of a single faceted conical tip. Still further, the tip portion 36 could be configured as a blunt tip, as shown for example in FIG. 12.

With reference to FIGS. 6 and 9, to assemble the stylet 20, the shank 38 of the tip body 24 is press fit and/or welded into a corresponding groove or slot 42 residing on or about the distal portion of the stylet body 22. Once assembled, a weld region 44 or, if welding is not performed, a region of frictional contact 46 at the interface of the stylet shank 38 and the outer surfaces of the stylet slot 42 is formed. It should be noted, however, that the stylet body 22 and tip body 24 could be assembled by other methods known to those of ordinary skill in the art. For example, the bodies 22, 24 could be bonded together by an adhesive or bonding material, or could be mechanically attached by a screw or rivet. Once assembled, the stylet body 22 and tip body 24 form the complete stylet 20 and are prevented from moving relative to each other to avoid detachment during a biopsy procedure. Although the stylet 20 is generally illustrated as a multiple component assembly, it should be noted, however, that the stylet 20 could alternatively be manufactured as a single piece, as shown, for Example in FIG. 13. In this embodiment, a separate tip body 24 is not required, as the stylet body 22 incorporates the entire tip body; hence, assembly is not required. The single piece stylet 20 could be made in its entirety from a single blank (i.e. from a single work piece) using any of numerous manufacturing processes and techniques known to those of ordinary skill in the art. Or, for example, the stylet could be molded as a single part using any of numerous molding processes and techniques known to those of ordinary skill in the art.

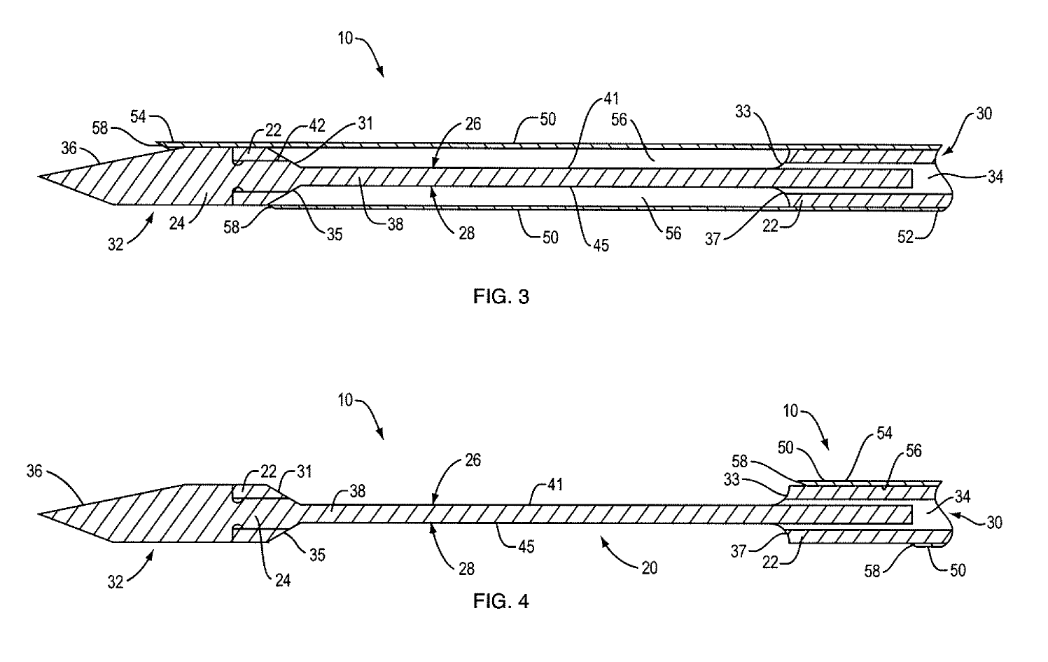

As noted above, and with particular reference to FIGS. 1 and 3, the needle set 10 further comprises a cannula 50 defining open proximal and distal ends 52, 54 and a lumen 56 (see, FIGS. 3-5) extending therebetween. The lumen 56 is sized and otherwise configured so that the cannula 50 slidably mounts over the stylet 20, allowing the cannula 50 and stylet 20 to move relative to one another between respective first (retracted) and second (extended) positions. The cannula 50 further includes a cutting edge 58 formed on its open distal end 54. The cutting edge 58 is sharpened or otherwise adapted to sever tissue prolapsing into the tissue receiving openings 26, 28 upon contact, as the cannula moves or slides in the direction from its first (retracted) position (FIG. 2) to its second (extended) position (FIG. 1). Further, the spatial relationship (both axially and radially) of the cannula 50 with respect to the stylet 20 and, in particular, with respect the tissue receiving openings 26, 28, is such that the cannula 50 is adapted to sever tissue residing in the tissue receiving openings substantially simultaneously in a single cutting stroke, thereby obtaining at least two biopsy cores in a single cutting stroke.

The needle set described herein is typically used with a spring loaded core (SLC) biopsy device or a vacuum assisted spring loaded core biopsy device (VASLC), examples of which are described in U.S. Pat. No. 7,390,306 and U.S. Patent Application Publication Nos. 2008/0200833 and 2009/0082696, which are incorporated by reference in their entireties as part of the present disclosure. However, without limitation, the needle set 10 can be used on other biopsy devices, for example, biopsy devices driven and/or actuated by electric, electro-magnetic, pneumatic and/or hydraulic motors, or by any other mechanical, electrical, magnetic, electronic and/or electro-mechanical drive units in addition to those described herein.

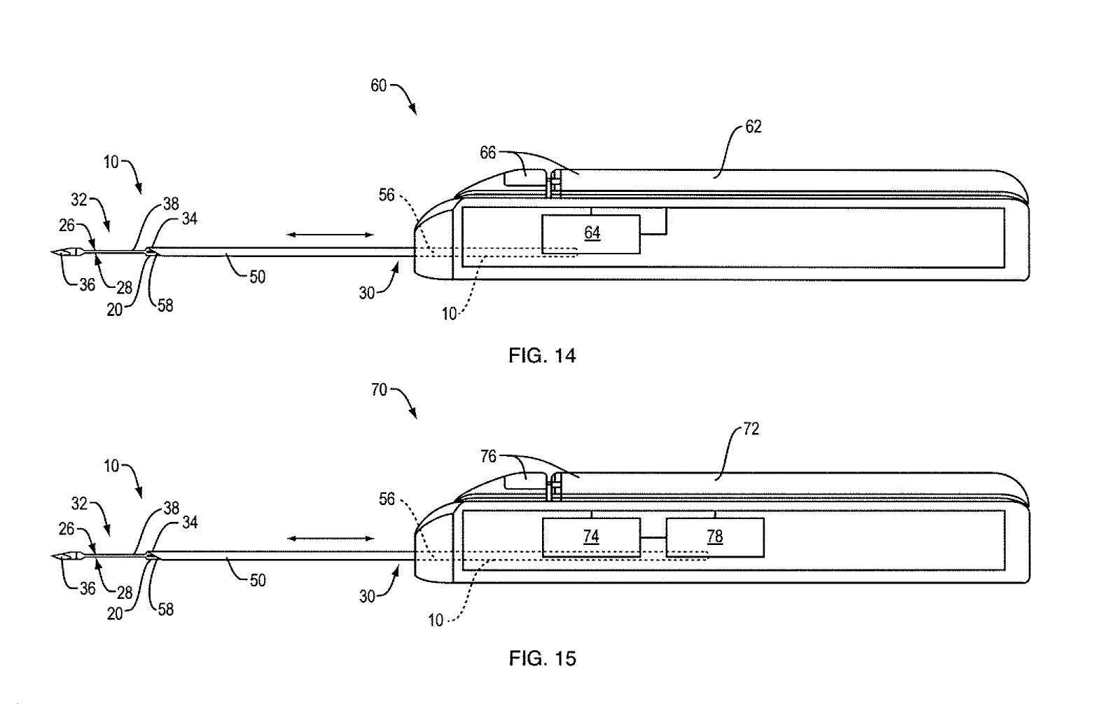

Referring now to FIGS. 14-15, the needle set 10 is shown mounted to an SLC biopsy device 60 (FIG. 14) and a VASLC biopsy device 70 (FIG. 15), each including (at least) a respective housing 62, 72 and drive mechanism 64, 74. The housings 62, 72 are typically configured so that the corresponding biopsy devices are hand-held when operated; however, it should be noted that the devices could be emplaced on or about a support or fixture and operated therefrom if desired. Once assembled to their respective housing 62 or 72, both the stylet 20 and cannula 50 are independently movable relative to said housing 62 or 72 between their respective first (retracted) and second (extended). Internally, the drive mechanisms 64, 74 are operatively connected to the needle set 10 to selectively move their corresponding stylet 20 and cannula 50 between their respective first (retracted) and second (extended) positions. Further, the drive mechanisms 64, 74 are configured to fire their respective cannula 50 in a single cutting stroke from the first cannula position (FIG. 2) toward the second cannula position (FIG. 1), substantially simultaneously severing tissue residing in the tissue receiving openings 26, 28 to obtain at least two biopsy cores (i.e. at least one core in each opening).

Typically, the drive mechanisms 64, 75 are spring-loaded mechanisms employing the forces supplied by a single spring or a combination of springs (not shown) to move and fire the cannula 50 and/or stylet 20 axially relative to the longitudinal axis of the device. The spring or springs are mechanically coupled (directly and/or indirectly) to the cannula 50 and/or stylet 20 in any of numerous ways known to those of ordinary skill in the art to effectuate the desired movements of the cannula 50 and stylet 20. For example, in one configuration, the spring or springs are mechanically coupled (directly and/or indirectly) to one or more carriages or carrier members (not shown), which in turn are mechanically coupled (directly and/or indirectly) to the cannula 50 and/or stylet 20 in such a manner that any or all spring forces applied (directly and/or indirectly) to the respective carrier members generate the desired movements of the cannula and/or stylet along the longitudinal axis of the respective biopsy device. As recognized by those of ordinary skill in the art, the spring or springs incorporated into the drive mechanisms 64, 75 can be any of numerous types of springs such as, for example, coil springs (constant or variable pitch), leaf springs or torsion springs. Further, if desirable, any or all of the springs used in the drive mechanisms could be replaced by other known biasing members, including but not limited to, elastic or elastomeric biasing members.

To operate the respective drive mechanisms 64, 74, the devices 60, 70 further comprise one or more actuators 66, 76. The actuators 66, 76, which are typically manually engageable, are operatively connected and/or linked to their respective drive mechanism 64, 74 to selectively engage and actuate said mechanisms to, in turn, selectively move the stylet 20 and/or cannula 50. In some embodiments, the actuators 66, 76 include a combination of one or more manually engageable levers and/or depressible members, which are mechanically linked and/or coupled (directly and/or indirectly) to their respective drive mechanisms 64, 74. In one such embodiment, for example, the actuators 66, 76 include a lever pivotable at one end about an axis and movable between a first lever position and a second lever position; a first depressible member releasably coupled to the lever, and movable between a first position and a second position to lock and unlock the lever; a second depressible member movable between a first position and a second position to effectuate firing of the stylet (i.e. effectuate the distal movement of the stylet relative to the device housing); and a third depressible member movable between a first position and a second position to effectuate firing of the cannula (i.e. effectuate movement of the cannula between its retracted position (FIG. 2) and its extended position (FIG. 1) relative to the stylet). Typically, the lever and depressible members are biased towards their respective first positions; however, such biasing is neither absolute nor required and should not be interpreted in the limiting sense. Also, it should be noted that any of the actuators employed could be rotated (as opposed to being depressed or pivoted) in a clockwise or counterclockwise motion to operate the respective drive mechanisms; hence, the type of motion required to move the actuators should not be interpreted in a limiting sense.

Drawing particular attention to FIG. 15, the VASLC biopsy device further includes a vacuum source 78 in communication with the at least two tissue receiving openings 26, 28 of the stylet 20. Although the vacuum source 78 is shown as being internal (i.e. an internal vacuum source residing within the housing 72), it should be noted that the vacuum source 78 could be an external vacuum source remote from the housing. During a biopsy procedure, vacuum generated by the vacuum source 78 is delivered to the tissue receiving openings 26, 28 to draw tissue into the tissue receiving openings and/or maintain the tissue within the tissue receiving openings, while the cannula 50 severs the tissue. With the assistance of vacuum, the probability of obtaining denser, better quality tissue cores is increased. In one embodiment, the vacuum generated by the vacuum source 78 is delivered to the tissue receiving openings 26, 28 through the stylet lumen 34; however, vacuum could be delivered to the openings 26, 28 by other means such as through ample spacing (if provided) at the interface of the stylet 20 and cannula 50 (e.g. between the outer surface of the stylet 20 and the inner surface of the cannula lumen 56). To control vacuum generation, the actuator 76, which operates the drive mechanism 74, may be employed such that a particular movement of the actuator 76 will start and/or terminate vacuum generation and/or allow the vacuum to vent to the atmosphere. However, it should be noted that a separate actuator or control device could be employed to control the vacuum source 78 separately from the drive mechanism 74.

Having thus described the needle set 10, its components and related biopsy devices 60, attention will now be drawn to a method of performing a biopsy to harvest multiple biopsy cores using said needle set and biopsy devices. Given a predetermined target site (not shown), the needle set 10 (mounted to either biopsy device 60 or 70) is introduced to the target site and the tissue receiving openings 26, 28 are exposed. To expose the tissue receiving openings 26, 28, the stylet 20 is typically fired distally relative to the cannula 50; however, it is also possible to retract the cannula 50 relative to the stylet 20 depending on the device configuration. In some cases, the stylet 20 is fired outside of the patient to expose the openings, and in other cases the stylet 20 is fired within the patient in proximity of the target site. Whether to fire the stylet inside or outside of the patient is determined by the residing physician on a case by case basis, factoring in, for example, the physician's personal preference or comfort level, the size of the patient's breasts (if a breast biopsy is being performed) or the location of the target site. Having exposed the tissue receiving openings 26, 28 to surrounding target tissue and allowing the surrounding tissue to prolapse into the tissue receiving openings 26, 28, the cannula 50 is advanced or fired distally. More specifically, the cannula 50 is advanced or fired in a single cutting stroke from its first (retracted) position (shown in FIG. 2) toward its second (extended) position (shown in FIG. 1) and over the tissue receiving openings 26, 28. This action substantially simultaneously severs tissue residing in the openings 26, 28 from the surrounding target tissue, leaving a biopsy core (not shown) in each opening for retrieval and analysis.

Once the biopsy cores are obtained, the device is removed from the patient and the cannula 50 is then retracted (or the stylet 20 is advanced depending on the arrangement) to expose the tissue receiving openings 26, 28. With the openings 26, 28 exposed, the cores are removed therefrom and placed in a container for storage, until the cores are removed for analysis. The method is then repeated as desired until a desirable number of cores are obtained.

If a VASLC biopsy device is used, the method further comprises the steps of generating vacuum via the vacuum source 78, and delivering vacuum to the tissue receiving openings 26, 28 to draw in and maintain tissue within the tissue receiving openings 26, 28. At a minimum, the vacuum is delivered while the cannula 50 severs tissue during the cutting stroke; however vacuum can be delivered in advance of the cutting stroke and can remain in the system thereafter until it is vented.

The needle sets and biopsy devices described herein can be provided in any suitable shape and size and can be manufactured using any suitable materials known to those of ordinary skill in the art. For example, in one particular embodiment, the needle set (stylet and cannula) is composed of surgical grade stainless steel, and the biopsy device housings and other components are composed of polymeric materials.

As may be recognized by those of ordinary skill in the pertinent art based on the teachings herein, numerous changes and modifications may be made to the above-described and other embodiments of the present invention without departing from the spirit and scope of the invention as defined in the specification, drawings and appended claims. It should be understood that the embodiments shown and described and all changes and modifications that come within the spirit and scope of the invention are desired to be protected. Accordingly, this disclosure is to be taken in an illustrative, as opposed to a limiting sense.

What is claimed is:

1. A needle set for a biopsy device comprising:
a stylet movable between a first stylet position and a second stylet position, the stylet defining an open proximal end, at least two tissue receiving openings at a distal end thereof and a lumen extending from the open proximal end to the at least two tissue receiving openings, wherein the tissue receiving openings are in fluid communication with the lumen; and
a cannula movable between a first cannula position and a second cannula position, the cannula including an open distal end with a cutting edge formed thereon, wherein the cannula is slidably mounted over the stylet such that the cannula and stylet are movable relative to one another between their respective first and second positions,
wherein the cannula is adapted to sever tissue residing in the tissue-receiving openings in a single cutting stroke from the first cannula position toward the second cannula position to obtain at least two biopsy cores.

2. A needle set as defined in claim 1, wherein the stylet includes a tissue piercing tip or a blunt tip at its distal end.

3. A biopsy device as defined in claim 1, further comprising a vacuum source in communication with the at least two tissue receiving openings, wherein vacuum generated by the vacuum source is delivered to the tissue receiving openings to at least one of (I) draw tissue into the tissue receiving openings and (ii) maintain the tissue within the tissue receiving openings, while the cannula severs the tissue.

4. A needle set as defined in claim 3, wherein vacuum generated by the vacuum source is delivered to the tissue receiving openings through the lumen.

5. A needle set as defined in claim 1, wherein the stylet and cannula are operatively connected to a drive assembly, which selectively moves the stylet and cannula between their respective first and second positions.

6. A needle set as defined in claim 1, wherein the stylet defines two diametrically opposed tissue receiving openings.

7. A needle set as defined in claim 1, wherein the tissue-receiving openings include a pair of opposing end sections and a pair of side sections extending longitudinally along the stylet between the end sections, and at least one of (i) at least one end section and (ii) at least one side section defines a plurality of protrusions adapted to engage tissue residing in the tissue-receiving openings.

8. A biopsy device, comprising:
a needle set mounted to a housing, the needle set comprising:
a stylet movable relative to the housing between a first stylet position and a second stylet position, the stylet defining an open proximal end, at least two tissue receiving openings at a distal end thereof and a lumen extending from the open proximal end to the at least two tissue receiving openings, wherein the tissue receiving openings are in fluid communication with the lumen; and
a cannula movable relative to the housing between a first cannula position and a second cannula position, the cannula including an open distal end with a cutting edge formed thereon, wherein the cannula is slidably mounted over the stylet such that the cannula and stylet are movable relative to one another between their respective first and second positions; and
a drive mechanism operatively connected to the needle set to selectively move the stylet and cannula between their respective first and second positions, wherein the cannula is adapted to sever tissue residing in the tissue-receiving openings in a single cutting stroke from the first cannula position toward the second cannula position to obtain at least two biopsy cores.

9. A biopsy device as defined in claim 1, further comprising a vacuum source in communication with the at least two tissue receiving openings, wherein vacuum generated by the vacuum source is delivered to the tissue receiving openings to at least one of (i) draw tissue into the tissue receiving openings and (ii) maintain the tissue within the tissue receiving openings, while the cannula severs the tissue.

10. A biopsy device as defined in claim 9, wherein vacuum generated by the vacuum source is delivered to the tissue receiving openings through the lumen.

11. A biopsy device as defined in claim 8, wherein the stylet includes a tissue piercing tip or a blunt tip at its distal end.

12. A biopsy device as defined in claim 8, further comprising a manually engageable actuator operatively connected to the drive mechanism, the actuator adapted to selectively actuate the drive mechanism to, in turn, selectively move the stylet and cannula.

13. A biopsy device as defined in claim 9, further comprising a manually engageable actuator operatively connected to the drive mechanism, the actuator adapted to selectively actuate the drive mechanism to, in turn, at least one of (i) selectively move the stylet and cannula and (ii) generate vacuum.

14. A biopsy device as defined in claim 9, wherein the vacuum source is at least one of an internal vacuum source and an external vacuum source.

15. A biopsy device as defined in claim 8, wherein the stylet defines two diametrically opposed tissue receiving openings.

16. A needle set as defined in claim 8, wherein the tissue-receiving openings include a pair of opposing end sections and a pair of side sections extending longitudinally along the stylet between the end sections, and at least one of (i) at least one end section and (ii) at least one side section defines a plurality of protrusions adapted to engage tissue residing in the tissue-receiving openings.

17. A method of harvesting multiple biopsy cores from a predetermined target site within a patient
comprising:
providing a needle set comprising a stylet including an open proximal end, at least two tissue receiving openings at a distal end thereof and a lumen extending from the open proximal end to the at least two tissue receiving openings, and a cannula defining an open distal end with a cutting edge formed thereon, wherein the cannula is slidably mounted over the stylet such that the cannula and stylet are movable relative to one another;
introducing the needle set to the target site and exposing the tissue receiving openings to surrounding target tissue and causing surrounding tissue to prolapse into the tissue receiving openings; and
advancing the cannula in a single cutting stroke over the tissue receiving openings and substantially simultaneously severing tissue leaving a biopsy core in each tissue receiving opening.

18. A method as defined in claim 17, further comprising:
generating vacuum; and
delivering vacuum to the tissue receiving openings to draw in and maintain tissue within the tissue receiving openings while the cannula severs tissue.

19. A method as defined in claim 17, wherein during the step of introducing the needle set to the biopsy site, the stylet is fired distally relative to the cannula to expose the tissue receiving openings.

20. A method as defined in claim 19, wherein (i) the stylet is fired outside of the patient or (ii) the stylet is fired within the patient in proximity of the target site.
